(19) United States
(12) Patent Application Publication
(10) Pub. No.: US 2012/0245512 A1
(43) Pub. Date: Sep. 27, 2012

(54) NON-LINEAR FREE STATE CATHETER

(75) Inventors: Thomas Wade Fallin, Hyde Park, UT (US); Jean-Sebastien Merette, Chester Springs, PA (US); Patrick Michel White, West Chester, PA (US); Alan Chervitz, Palm Harbor, FL (US); Cary R. Reeves, Broomfield, CO (US)

(73) Assignee: VITAL 5, LLC, North Logan, UT (US)

(21) Appl. No.: 13/240,624

(22) Filed: Sep. 22, 2011

Related U.S. Application Data
(60) Provisional application No. 61/385,309, filed on Sep. 22, 2010.
(60) Provisional application No. 61/450,089, filed on Mar. 7, 2011.
(60) Provisional application No. 61/494,805, filed on Jun. 8, 2011.

(51) Int. Cl. A61M 25/14; A61M 1/00; A61M 25/08

(52) U.S. Cl. 604/28; 604/523; 604/500; 604/528; 604/43

(57) ABSTRACT

Devices and methods are disclosed to conduct fluid away from or deliver fluid to an area of a treatment site of a patient's body. For example, a catheter is provided having a terminal end with a non-linear shape such that fluid is delivered to or drained from an area of a treatment site.

### CROSS-REFERENCE TO RELATED APPLICATIONS

This application claims the benefit of U.S. Provisional Application No. 61/385309, filed Sep. 22, 2010; U.S. Provisional Application No. 61/450,089, filed Mar. 7, 2011; and U.S. Provisional Application No. 61/494,805, filed Jun. 8, 2011.

### FIELD OF THE INVENTION

The invention relates to devices and methods for transporting fluid to or from a treatment site of a patient's body.

### BACKGROUND

Many medical procedures benefit from transporting fluid to or from a treatment site of a patient's body. Devices for transporting fluid in a medical procedure are generally referred to as catheters. They may be used to provide drainage or administer treatment fluids. For example, catheters may be used to drain fluids from organs or from areas of abnormal fluid collection such as in a surgical wound following a surgical procedure. Catheters may also be used to deliver fluid to a treatment site to provide a vast range of therapies from cancer treatment to nutritional supplementation. A few exemplary therapies include stimulating tissue growth, administering antibiotics, flushing away impurities, killing or halting the reproduction of cancer cells, and relieving pain.

Catheters may be used in gravity driven arrangements such as with a collection container located below the treatment site or a medication container located above the treatment site. Likewise, catheters may be used in pressurized arrangements. For example, suction may be applied to a drainage catheter to draw fluids away from the treatment site. Suction devices may include elastomeric bulbs, spring actuated bellows, electromechanical vacuum pumps, and other known medical suction devices. Pressurized fluid may also be delivered through a catheter to the treatment site. For example, fluid infusion devices may include manual syringes, elastomeric infusion devices, spring loaded infusion devices, electromechanical infusion devices, and other known infusion devices.

Typical prior catheters are linear devices having one or more openings formed along a portion of their length through which fluid passes. They often perform poorly due to an inability to drain fluids from or deliver fluids to a sufficiently large area to encompass the entire treatment site. In addition, tissue folds and tissue apposition further affect the movement and collection of fluid making it difficult for prior catheters to adequately address the treatment site.

For example, where a treatment site encompasses a two or three dimensional treatment area, prior drainage catheters are only able to drain fluid from a relatively small, linear portion of the treatment area often leaving behind pockets of fluid.

Similarly, prior infusion catheters only deliver treatment fluid to a relatively small, linear portion of the treatment site leaving much of the site untreated. Prior infusion catheters may also deliver too much treatment fluid to a relatively small area resulting in pooling of treatment fluid or contact with non-target tissues. For example, infusion catheters may be used to deliver pain relieving medication directly to a surgical site to provide, for example, post-operative relief of pain resulting from a surgical intervention. If the medication does not reach tissue disrupted during the surgical intervention, it may not relieve the pain. Alternatively, if the medication is delivered indiscriminately, undesired interactions may occur with local structures such as, for example, spinal nerves or vital organs.

### SUMMARY

Aspects of the invention provide devices and methods to conduct fluid away from or deliver fluid to an area of a treatment site of a patient's body. Fluid delivered to a treatment site will be referred to as treatment fluid and may be any material delivered to the treatment site to obtain a desired effect. For example treatment fluid may be water, saline, antibiotics, antiviral agents, hormones, growth factors, anti-inflammatories, analgesics, anesthetics, and/or any other material useful in treating a patient. For example, anesthetics may include marcaine, rupivicaine, bupivacaine, and/or any other anesthetic or combinations thereof.

Embodiments of the invention may be used in any procedure in which it is desirable to deliver fluid to or remove fluid from a treatment site. The use of such devices and methods will be described with some specific examples illustrating the delivery of an anesthetic to a surgical site for the treatment of post-operative pain and/or removal of fluids from a surgical site. Examples of such surgical procedures include surgery of the head, neck, chest, back, abdomen, and the extremities. Examples include general surgery, cosmetic surgery, joint surgery, and spine surgery. However, it will be apparent to one having skill in the art that the disclosed devices and methods may be used to treat a variety of other conditions by drainage of fluids from and delivery of fluids to a treatment site.

In one aspect of the invention, a catheter includes a first or connection end and an opposite, second or terminal end. A fluid conduit extends between the connection end and the terminal end for passing fluids. The terminal end includes an elongated hollow body having a wall defining the terminal end of the fluid conduit and one or more openings formed through the wall for passage of fluid between the fluid conduit and an exterior of the conduit. The terminal end may describe a non-linear path such that fluid is delivered to or drained from an area generally within a plane. The terminal end may describe a non-linear path such that fluid is delivered to or drained from a three-dimensional area. The terminal end may be flexible to permit adjustment of the coverage area of the catheter. Such adjustment may be accomplished within a plane by moving portions of the terminal end relative to one another to cover varying areas. Such adjustment may be accomplished in three dimensions by moving portions of the terminal end relative to one another to cover varying non-planar areas. The non-linear configuration of the terminal end results in portions of the terminal end separating tissue layers at the treatment site and maintaining fluid communication between the tissue layers over a two-dimensional or three-dimensional treatment site to extend the effective treatment area. Furthermore, the non-linear configuration extends peripherally into the tissue folds and irregularities to separate tissue layers and enhance fluid transport between the layers and adjacent the terminal end. Enhancement of fluid transport reduces the number of catheters required to transport fluid to and/or away from the treatment site. The one or more openings in the wall of the conduit may be positioned at any circumferential position around the wall. They may be placed parallel to the plane of the non-linear path of the terminal end so that they open within the space between tissue layers to avoid blocking of the openings by overlying tissue. The non-linear path of the terminal end may have a predetermined shaped that conforms to the margins of a particular surgical site. The shape may be polygonal, ovoid, spiral, or random shaped.

The terminal end may include more than one conduit. Multiple conduits may be adjustable two-dimensionally and/or three-dimensionally relative to one another to vary the fluid distribution pattern of the terminal end. Multiple conduits may provide for different functions. For example, one or more conduits may be used to infuse fluids to the treatment site while one or more other conduits may be used to drain fluids away from the treatment site.

The terminal end of the catheter may have a first non-linear configuration and a second configuration into which it may be modified. For example, the terminal end may have a non-linear deployed configuration for fluid transport to or from a two-dimensional or three-dimensional area and a delivery or removal configuration. The delivery or removal configuration may be smaller than the deployed configuration to ease placement or removal of the terminal end at a desired location of a patient's anatomy. For example, the delivery or removal configuration may be folded, rolled, straightened, stretched, compressed, twisted, deflated, and/or otherwise manipulated relative to the deployed configuration.

The catheter may be placed at the treatment site in an inside-out placement method in which it is placed in an open wound and the connection end is passed out of the patient's body leaving the terminal end at the treatment site. Alternatively, the catheter may be placed at the treatment site in an outside-in placement method in which the terminal end is introduced from outside the patient's body to the treatment site. Where a surgical incision is present near the treatment site, the catheter may extend through the incision. Alternatively, the catheter may extend through another opening, such as a stab incision, formed for the purpose of passing a portion of the catheter.

### BRIEF DESCRIPTION OF THE DRAWINGS

Various examples of the present invention will be discussed with reference to the appended drawings. These drawings depict only illustrative examples of the invention and are not to be considered limiting of its scope.

FIG. 1A is a perspective view of an embodiment of the invention;

FIG. 1B is a top plan view of the embodiment of claim 1A;

FIG. 1C is a cross-sectional view of the embodiment of claim 1A, taken along line C-C of FIG. 1B;

FIG. 2A is a perspective view of the embodiment of FIG. 1A, illustrating a method of installation;

FIG. 2B is a perspective view of the embodiment of FIG. 1A, illustrating a method of installation;

FIG. 2C is a perspective view of the embodiment of FIG. 1A, illustrating a method of installation;

FIG. 2D is a perspective view of the embodiment of FIG. 1A, illustrating a method of installation;

FIG. 2E is a perspective view of the embodiment of FIG. 1A, illustrating a method of installation;

FIG. 3A is a perspective view of an embodiment of the invention similar to FIG. 1A illustrating a shape variation;

FIG. 3B is a top plan view of an embodiment of the invention similar to FIG. 1A illustrating a shape variation;

FIG. 4A is a top plan view of an embodiment of the invention similar to FIG. 1A illustrating the inclusion of a second fluid conduit;

FIG. 4B is a top plan view of an embodiment of the invention similar to FIG. 1A illustrating the inclusion of a second fluid conduit;

FIG. 4C is a partial perspective view of an embodiment of the invention similar to FIG. 1A illustrating the inclusion of a second fluid conduit;

FIG. 5A is a perspective view of an embodiment of the invention similar to FIG. 1A illustrating a shape variation;

FIG. 5B is a perspective view of the embodiment of FIG. 5A illustrating the deployment of the embodiment;

FIG. 6A is a perspective view of an embodiment of the invention similar to FIG. 1A illustrating a shape variation; and

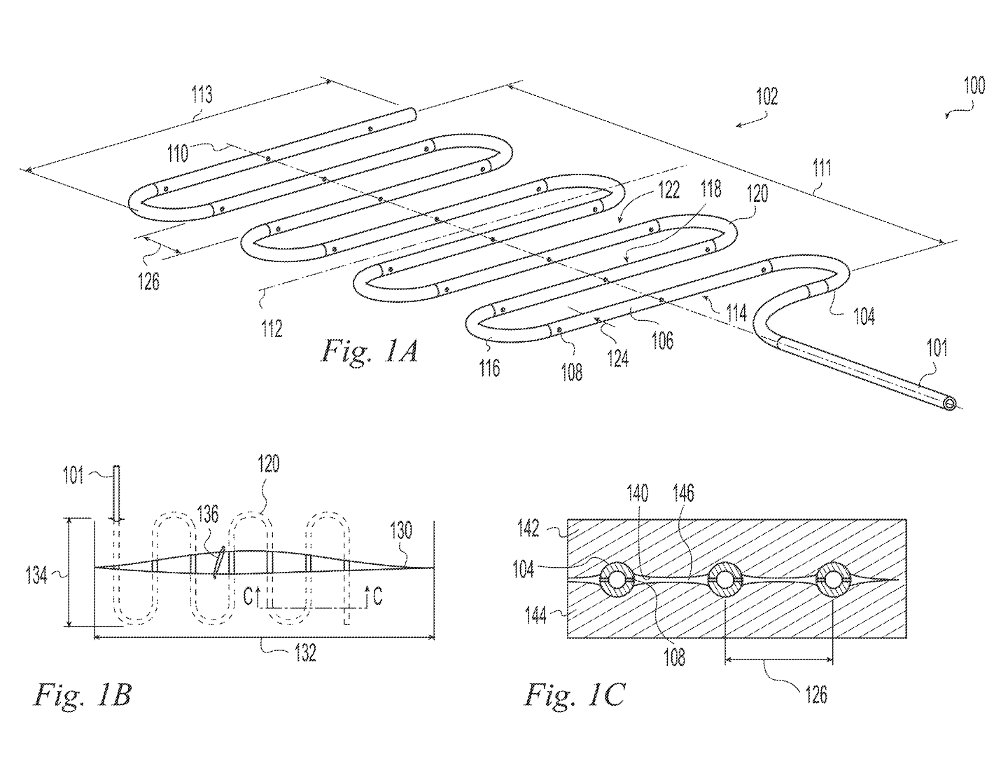

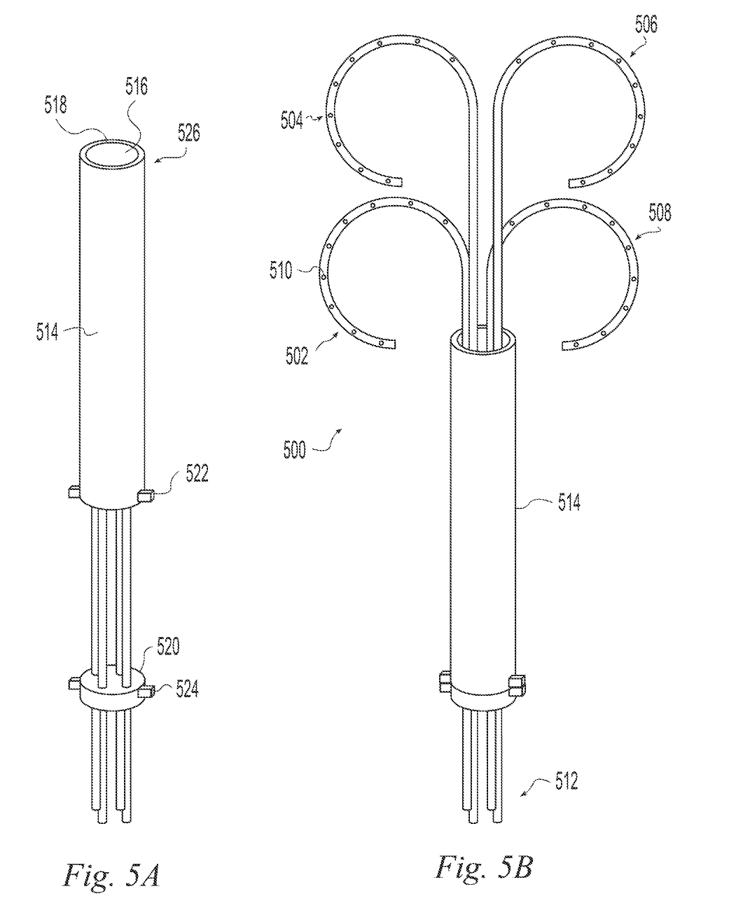

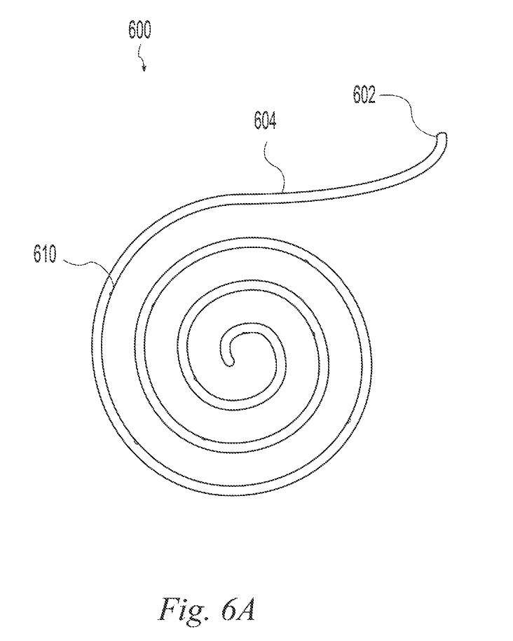

FIG. 5B is a perspective view of an embodiment of the invention similar to FIG. 1A illustrating a shape variation.

### DESCRIPTION OF THE ILLUSTRATIVE EXAMPLES

FIGS. 1A-C depict a catheter 100 having a connection end 101 and a terminal end 102. The terminal end 102 is in the form of an elongated fluid conduit 104 having a sidewall 106 and openings 108 communicating from the interior of the conduit 104 to the exterior of the conduit 104. The conduit 104 is formed into a non-linear original, or free state, shape for infusion or aspiration of fluid from a treatment site. The conduit 104 contains or is made of an elastic shape memory material so that it is biased toward and tends to return to the original, free state, non-linear shape but can be straightened or compressed for insertion and extraction. For example, the conduit 104 may be made of a heat settable elastic polymer allowing it to be formed into a non-linear, original, free state shape and then heated and cooled to lock-in the shape such that it is biased toward and tends to return to the heat-set shape. For example, the conduit may be made of or contain a thermoplastic elastomer such as a styrenic block copolymer, polyolefin, thermoplastic polyurethane, thermoplastic copolyester, thermoplastic polyamide, and/or their various blends. For example, the conduit may contain or be made of a polyether block amide or PEBA. PEBA is available from Arkema under the tradename of PEBAX®. In the illustrative example of FIG. 1, the conduit 104 is made of PEBA and is shaped into an original non-linear shape having a repeating back and forth two-dimensional pattern having a first directional axis 110 corresponding to the pattern length 111 and a second directional axis 112 corresponding to the pattern width 113. In the illustrative example of FIG. 1, a generally straight first portion 114 of the conduit crosses the length axis 110 generally perpendicular to the axis. A first bend 116 connects the first portion to a generally straight second portion 118 generally parallel to the first portion 114 which also crosses the length axis 110 generally perpendicular to the axis. A second bend 120 connects the second portion to a generally straight third portion 122 generally parallel to the second portion 118 which also crosses the length axis 110 generally perpendicular to the axis. Any number of portions may be connected in this manner to create a terminal end 102 of a desired size. The illustrative pattern of FIG. 1 may be described as a serpentine or zigzag shape comprising a regular repeating pattern of two or more curvilinear segments. In particular, the illustrative pattern of FIG. 1 forms a continuous path comprised of different curvilinear segments that are bounded by a perimeter defined by the length 111 and width 113, the segments being spaced generally equal distances from one another.

In the illustrative example of FIG. 1, the sidewall 106 has a first dimension 124 generally parallel to the length dimension. For a conduit 104 having a tubular shape, the first dimension corresponds to the diameter of the conduit. The spacing 126 between adjacent portions in the original non-linear shape may vary from 1 to 100 times the first sidewall dimension. More particularly, the spacing may vary from 1 to 20 times the first sidewall dimension. Still more particularly, the spacing may vary from 2-5 times the first sidewall dimension.

The openings 108 may be located in the generally straight portions and excluded from the bends to help prevent kinking of the conduit 104 that might occur if the openings 108 were located in the bends.

The illustrative terminal end 102 may be placed in a treatment site as shown in FIGS. 1B-1C. A surgical incision 130 provides access to a treatment site 140 between overlying tissue layers 142, 144. The terminal end 102 is placed between the tissue layers 142,144 to drain or infuse the treatment site. The conduit 104 tends to create and/or maintain folds or separations 146 between the tissue layers 142, 144 to facilitate fluid flow through openings 108 and along the length 132 and width 134 of the treatment site. The spacing 126 of the segments, the diameter of the conduit 104, and the length 111 and width 113 of the terminal end all contribute to the coverage are of the terminal end 102. The openings 108 may be positioned at any circumferential position around the conduit 104. In the illustrative example of FIG. 1, they are positioned parallel to the plane of the two dimensional pattern so that they open within the space between tissue layers to avoid blocking of the openings by overlying tissue. This opening 108 orientation is best seen in FIG. 1C.

The incision 130 may be closed temporarily or permanently with a fastener 136. The fastener may be a staple, suture, surgical adhesive, butterfly closure, or other suitable fastener. One or more fasteners may be placed at the skin surface, subcutaneously, intrafascially, intramuscularly, or otherwise.

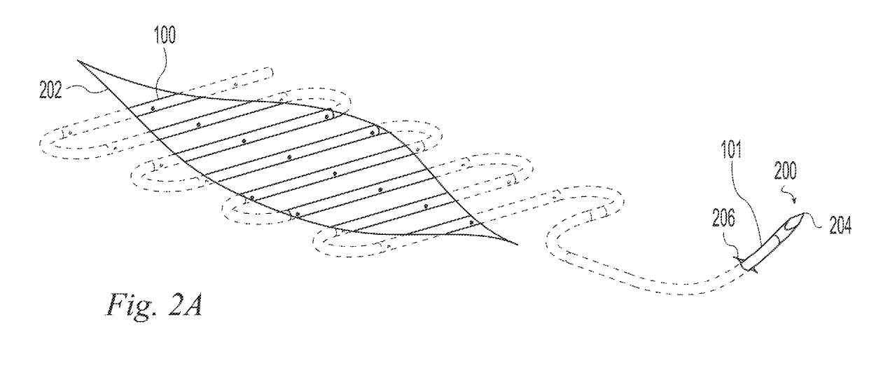

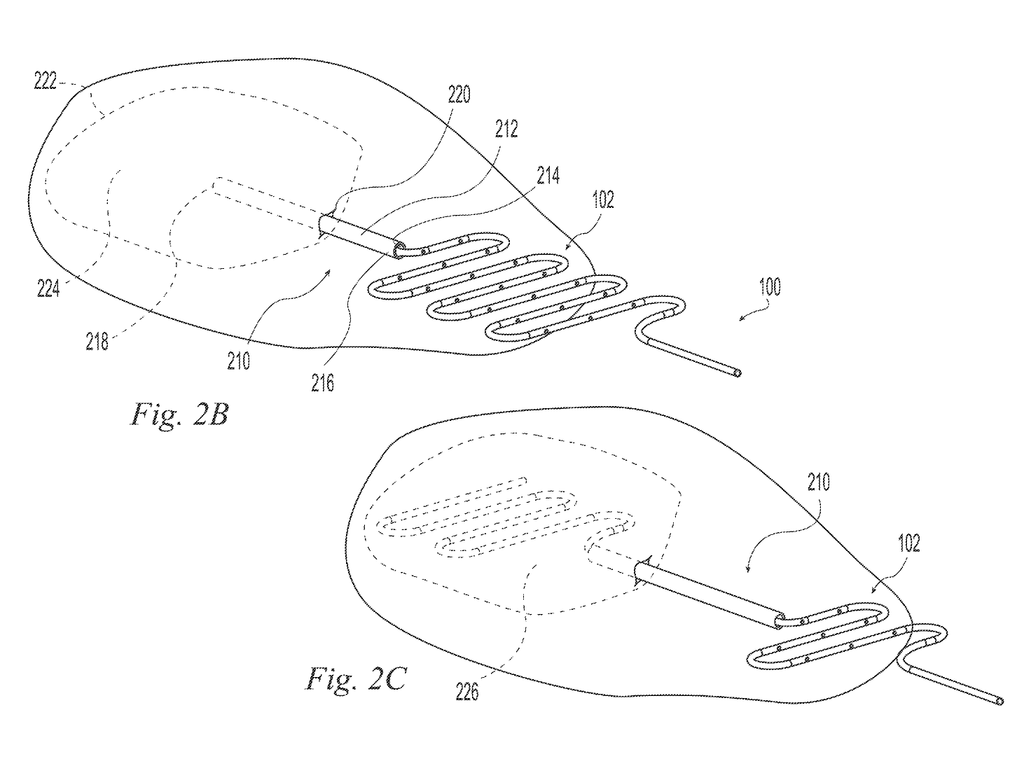

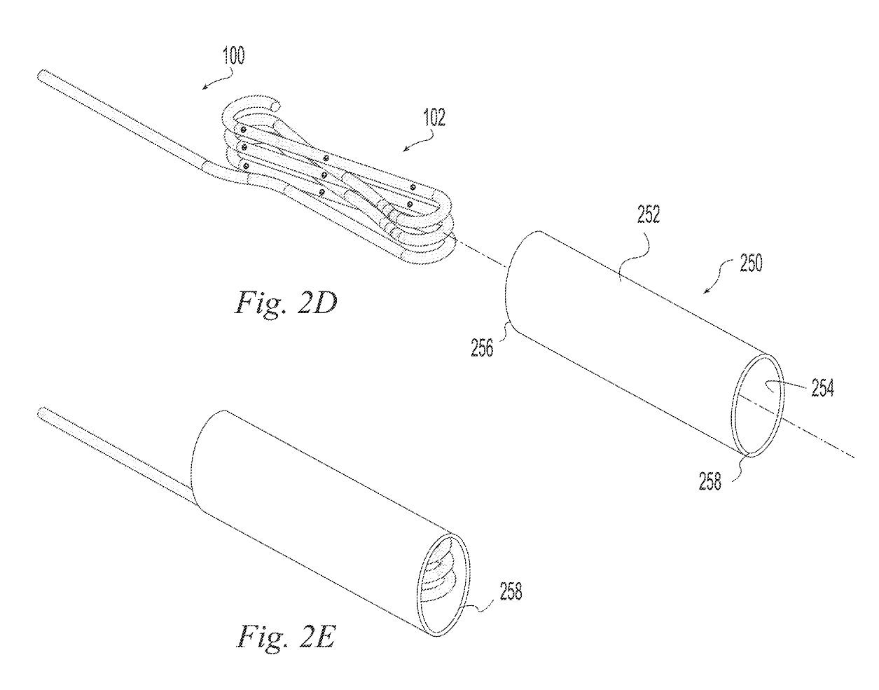

FIG. 2 illustrate devices and methods for introducing the catheter 100 of FIG. 1 into a treatment site. FIG. 2A illustrates an introducer in the form of a trocar 200 connectable to the connection end 101 of the catheter 100 in order to pass the connection end 101 out of the patient in an inside-out placement method. For example, during a surgical procedure having an incision 202 to permit access to a surgical site, the catheter 100 may be placed before the incision is closed. The trocar 200 is connected to the connection end 101 such as by insertion of the connection end 101 into a bore in the trocar 200 or inserting a barb extending from the trocar into the conduit of the connection end 101. The trocar 200 includes a sharp cutting tip 204 which is then passed from the surgical site through adjacent tissues and out of the skin through a puncture wound 206. The connection end 101 of the catheter 100 is pulled through the puncture wound 206 and the catheter 100 is positioned in the desired treatment site within the surgical insult. One or more layers of tissues are closed including the incision 202. The connection end 101 is connected to, for example, a fluid suction device or a fluid delivery device and treatment is initiated.

FIGS. 2B-2C illustrate an introducer 210 useable to pass the terminal end 102 of the catheter 100 into a patient to deliver the terminal end 102 to a treatment site 222 in an outside-in placement method. The introducer 210 in the illustrative example of FIGS. 2B-2C is in the form of a hollow tube or needle having a side wall 212 defining a lumen 214 extending from a proximal end 216 to a distal end 218. In use, a puncture wound 220 is created communicating from outside the patient through the patients tissues to the treatment site 222. If necessary, a space may be created by separating tissue layers at the treatment site by inserting and sweeping a probe into the treatment site. The distal end 218 of the introducer 210 is inserted through the puncture wound 220 and into the treatment site 222. In the illustrative example of FIG. 2B, the distal end 218 of the introducer 210 is inserted into a distal portion 224 of the treatment site 222. The terminal end 102 of the catheter 100 is fed through the introducer 210, straightening the bends 116, 120 as necessary so that it will pass through the lumen 214. As the terminal end 102 is delivered to the treatment site 222, the introducer may be withdrawn to help distribute the terminal end 102 throughout the treatment site 222 and allow it to return toward its original, free state. FIG. 2B shows the catheter 100 partially inserted with the introducer withdrawn partway from the distal portion 224 of the treatment site to near a proximal portion 226 of the treatment site 222.

FIGS. 2D-2E illustrate another introducer 250 useable to pass the terminal end 102 of the catheter 100 into a patient to deliver the terminal end 102 to a treatment site in an outside-in placement method. Like the introducer of FIGS. 2B-2C, the introducer 250 in the illustrative example of FIGS. 2B-2C is in the form of a hollow tube or needle having a side wall 252 defining a lumen 254 extending from a proximal end 256 to a distal end 258. The lumen 254 is sized to receive the terminal end 102 of the catheter 100 in a folded or collapsed state as shown in FIG. 2D in which the segments of the terminal end 102 are compressed to lie closely together. The compressed terminal end 102 may be pre-loaded into the introducer 250 prior to insertion of the distal end 258 of the introducer into the treatment site or the introducer 250 may be inserted first and then the terminal end 102 of the catheter loaded into the introducer 250. With the introducer 250 inserted at the treatment site, the terminal end 102 of the catheter 100 may be expelled from the distal end 258 of the introducer 250 and allowed it to return toward its original, free state.

The illustrative pattern of the terminal end 102 of FIG. 1 is bounded by a generally rectangular perimeter and in its original or free state is adapted to fit within a rectangular treatment site. The shape of the terminal end 102 may be changed intraoperatively to fit a desired treatment site by compressing, expanding, bending, stretching and/or otherwise manipulating the conduit 104 and fitting it within the treatment site where it will be constrained from returning to its original free state by the surrounding tissues. For example, to fit a longer or shorter treatment site, the terminal end 102 may be stretched or compressed along the length axis while maintaining its generally serpentine or zigzag shape. The shape of the terminal end 102 may be changed in three dimensions by moving the segments apart in a direction perpendicular to the first and second directional axes 110, 112.

The original non-linear shape may be varied, for example, by making the connecting bends smaller or larger, making the connected portions longer or shorter, angling the portions so that they are not generally perpendicular to the length axis, and/or making the shape of the connected portions themselves non-straight. In this way, the terminal end 102 may be formed with an original, free state, size and shape adapted to fit a particular wound geometry. The terminal end 102 may be provided in various configurations of length and width from which an appropriate size may be selected for a particular treatment site.

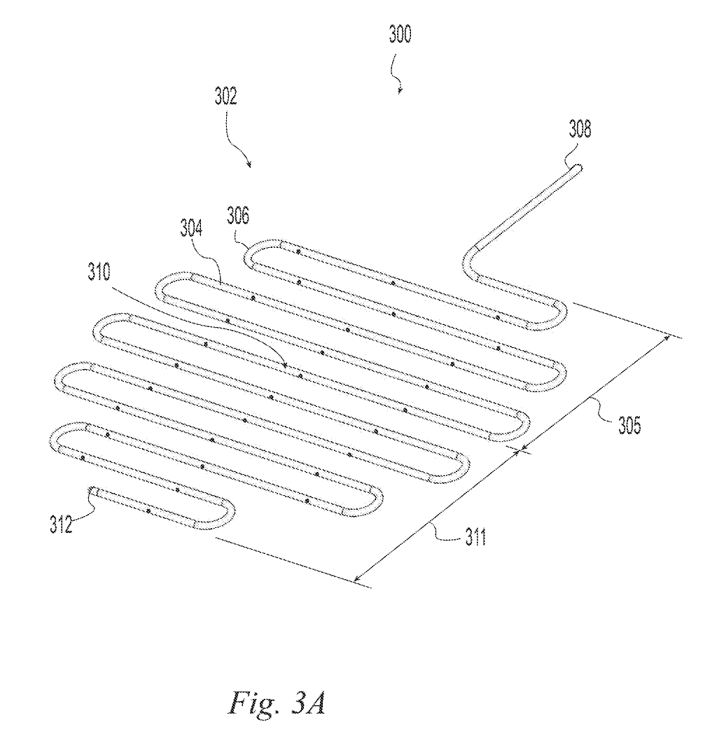

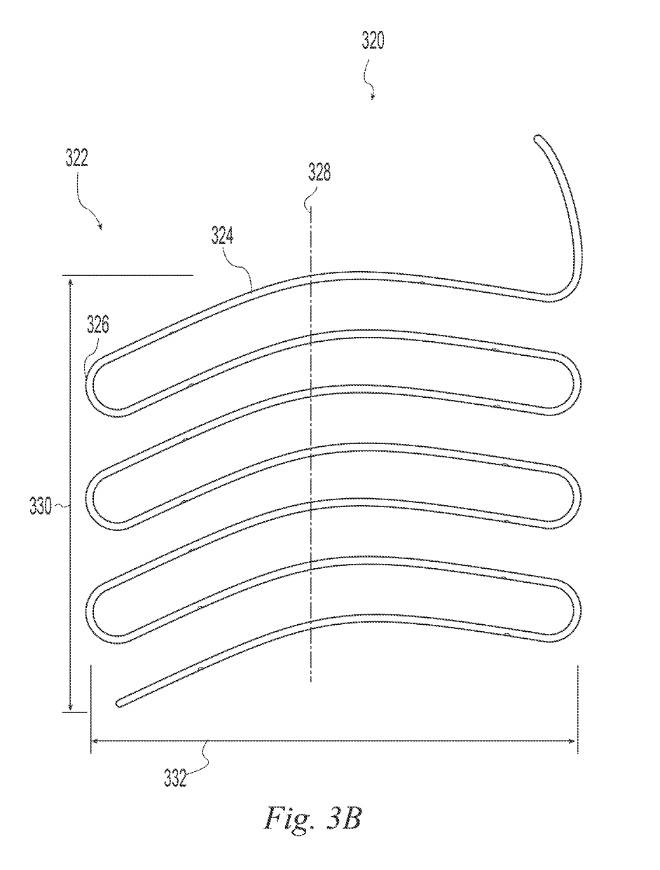

FIG. 3A illustrates a catheter 300 having a terminal end 302 with an alternate shape produced by varying the length of the generally straight portions 304 of the pattern interconnected by bends 306. A terminal end pattern may be produced that is bounded by a generally elliptical perimeter by increasing the length of each of the generally straight portions 304 in a first portion 305 of the pattern as they progress from near the connection end 308 toward the center 310 of the pattern and then decreasing the length of each of the generally straight portions 304 in a second portion 311 of the pattern as they progress from near the center 310 of the pattern toward the tip 312 of the terminal end 302. FIG. 3B illustrates a catheter 320 having a terminal end 322 with an alternate shape produced by replacing the generally straight portions with generally curved portions 324 to produce a non-linear shape having a repeating back and forth two-dimensional pattern of elongate curved segments 324 joined by bends 326. The pattern has a length 330 and a width 332. The bends have generally shorter radii 326 than the segments 324 and in the embodiment shown transcribe more than ninety degrees such that the segments 324 repeatedly cross the longitudinal axis 328. A terminal end pattern bounded by any desired shape may be produced by varying the size, shape, and spacing of the segments forming the terminal end 302.

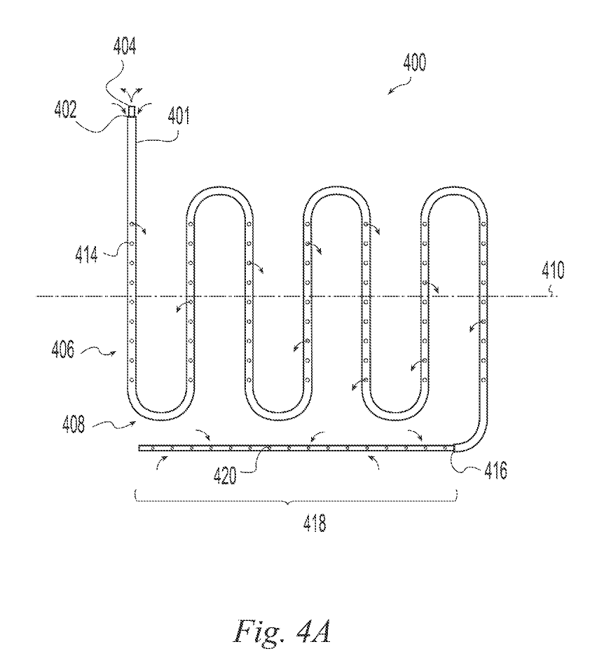

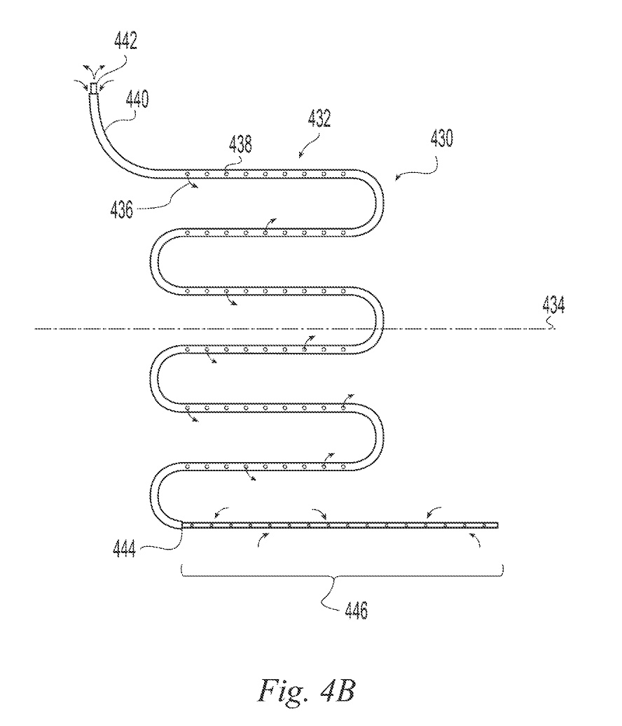

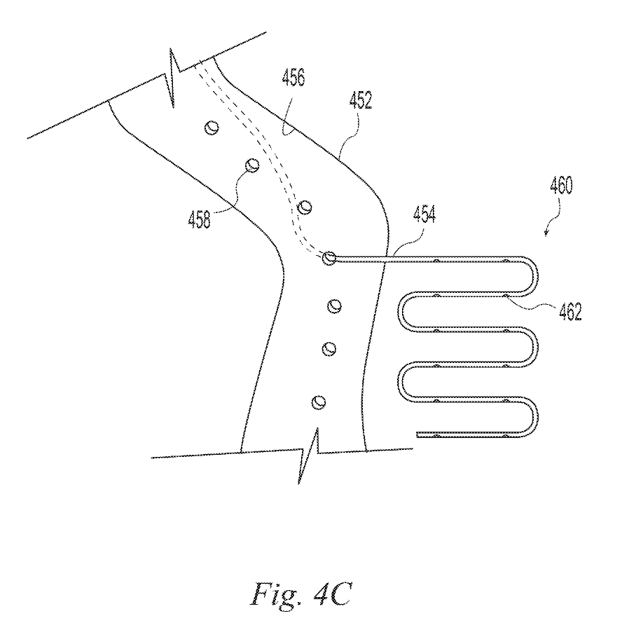

FIGS. 4A-C illustrate variations on the terminal end of FIG. 1. FIG. 4A depicts a catheter terminal end 400 having a dual conduit 401 including a first, infusion conduit 402 and a second, aspiration conduit 404. In the illustrative example of FIG. 4, the infusion and aspiration conduits 402, 404 are arranged coaxially with the aspiration conduit inside of the infusion conduit. Alternatively, for example, the conduits 402,404 could be side-by-side. The dual conduit includes straight portions 406 and bends 408 arranged like those in FIG. 1 with the straight portions generally perpendicular to a length axis 410. Treatment fluid 412 is delivered to the treatment site via openings 414 in the outer wall of the infusion conduit 402. The aspiration conduit 404 extends beyond the end 416 of the infusion conduit to form a fluid receiving portion 418. The fluid receiving portion 418 bends back in a direction generally perpendicular to the straight portions 406 to underlie the infusion portion of the terminal end 400. The fluid receiving portion 418 includes openings 420 for receiving treatment fluid for removal by the aspiration conduit 404. In use, the fluid receiving portion 418 is positioned at the treatment site where treatment fluid tends to pool and/or adjacent portions of the treatment site from which fluid is to be excluded. For example, the catheter terminal end may be positioned in a surgical wound, e.g. from a reconstructive surgical procedure such as breast or knee reconstruction procedure, in which treatment fluid, such as an anesthetic, can be advantageously applied over an extended area of disrupted tissue having a length and a width and in which the wound tends to drain to a lower portion of the treatment site. In another example, the fluid receiving portion 418 may be positioned adjacent to nerve roots to capture and transport away anesthetic fluid to protect the nerve root.

FIG. 4B depicts a terminal end 430 similar to that of FIG. 4A except that the straight portions 432 are generally parallel to a length axis 434. Treatment fluid 436 is delivered to the treatment site via openings 438 in the outer wall of the infusion conduit 440. The aspiration conduit 442 extends beyond the end 444 of the infusion conduit to form a fluid receiving portion 446. The fluid receiving portion 446 continues the pattern of bends and straight portions and extends generally parallel to the straight portions 432 to underlie the infusion portion of the terminal end 400.

FIG. 4C depicts an aspiration conduit 452 and an infusion conduit 454 in which the infusion conduit is located within an inner lumen 456 of the aspiration conduit 452 over a portion of its length. The aspiration conduit 452 includes openings 458 for aspirating fluid away from a treatment site. The infusion conduit 454 exits from the aspiration conduit lumen 456 via one of the openings 458 to form a terminal end 460 similar to those of FIGS. 4A and 4B having a repeating pattern of segments with openings 462 for infusing fluid to a treatment site.

FIGS. 5A-B depict a fluid delivery device in the form of a terminal end 500 of a catheter. The terminal end 500 includes one or more conduits 502, 504, 506, 508 having a deployed configuration with a non-linear original or free state such as, e.g. a curled shape or “pig tail”. The conduits 502-508 may be formed from shape memory tubing so that when deployed they tend to curl and cover an extended area but which allows the tubing to be straightened for deployment and/or extraction. Fluid openings 510 communicate fluid between the interior of the conduits and the treatment site. Multiple conduits may be joined at their connection ends 512 by a junction or manifold to a common supply line, not shown. While shown pre-formed into curled configurations, the conduits 502-508 may be formed into any suitable shape to transport fluid to or from a treatment area. For example, the conduits 502-508 may be formed into arcs, helixes, serpentine patterns, polygons of any number of sides, and/or any other two-dimensional or three-dimensional regular, irregular, or random shape. Any number of conduits may be used to provide a desired coverage pattern.

An introducer 514 may be used to aid in placing the conduits 502-508 at a treatment site. The introducer 514 has a relatively narrow, hollow interior 516 defined by a wall 518 that constrains the conduits 502-508 to a compact delivery configuration. Upon ejecting the conduits 502-508 from the introducer 514, they resume the expanded, deployed configuration of their original free state shape. In the illustrative example of FIG. 5 a tubular introducer is slidably disposed about the conduits 502-508. A retainer 520 joins the conduits 502-508 at their connection ends and may be coupled to them to fix their relative spacing both axially and radially. Alternatively, the retainer 520 may permit the conduits 502-508 to slide relative to the retainer 520 to permit adjustment of their relative positions. The introducer 514 includes a connector 522 and the retainer 520 includes a connector 524 cooperatively engageable with the introducer connector.

In use, the introducer 514 is placed in a first or delivery position as shown in FIG. 5A in which the shaped portions of the conduits 502-508 are constrained to a compact delivery configuration within the introducer. In this example, the conduits 502-508 are straightened inside the introducer 514. The delivery end 526 of the introducer 514 is positioned at a desired location relative to a treatment site and the conduits 502-508 are expelled from the introducer 514 such as by sliding the retainer 520 toward the introducer 514 and into the second or deployed position shown in FIG. 5B wherein the conduits expand into their deployed original, free state configuration. The conduits 502-508 may be fixed relative to the retainer to provide a fixed deployed configuration or they may be adjustable relative to the retainer either before or after deployment to tailor the deployed configuration. Alternatively, the retainer may be omitted and the conduits 502-508 manipulated individually within the introducer and treatment site. The connectors 522 and 524 may be engaged, such as by clamping, to fix the relative position of the introducer 514 and retainer 520. The introducer 514 and/or retainer 520 may be left in the patient's body or they may be removed leaving just the conduits 502-508. The introducer may be used as a guide to straighten the conduits 502-508 as they are removed.

The exemplary terminal end 500 of FIG. 5 advantageously expands to aspirate or infuse fluids over an expanded area while also having a relatively small extraction profile and force since the conduits 502-508 straighten upon being pulled away from the treatment site. Furthermore, the ability to fix the relative positioning of one conduit in relationship to another conduit allows for a selectable area of coverage.

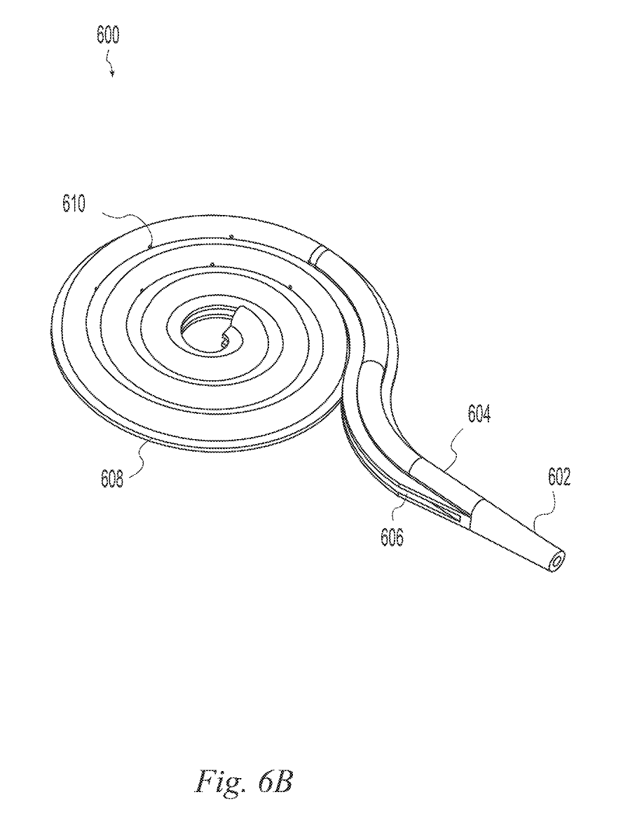

FIGS. 6A-B depict variations on the shape of a terminal end. FIG. 6A depicts a fluid delivery device in the form of a terminal end 600 of a catheter 602. The end 600 includes an elongated fluid conduit 604 formed into a coil with openings 610 that communicate fluid from the interior of the conduit 604 to the exterior while the coil separates tissue folds and provides a fluid delivery area within the tissue. The conduit 604 is formed from shape memory material so that it may be straightened for delivery and/or extraction but maintain the coiled configuration while deployed. FIG. 6B depicts a fluid delivery device similar to that of FIG. 6A but further having a groove 606 formed on a first side of the conduit 604 and a mating tongue 608 formed on an opposite side of the conduit 604. The conduit 604 is formed into a coil with the tongue 608 engaging the groove 606. The tongue and groove configuration creates a fluid barrier in the deployed condition yet allows the conduit 604 to be uncoiled during extraction to ease the extraction.

A treatment kit may be provided including one or more catheters according to various aspects of the invention. Optionally the kit may include an introducer. Optionally the kit may include an infusion source. Optionally the kit may include a drainage container.

What is claimed is:

1. An infusion catheter for delivering treatment fluid to a treatment site of a patient, the catheter comprising:
a first elongated fluid infusion conduit having a sidewall, the first conduit defining a fluid path from a location outside of the patient to the treatment site, the first conduit being flexible and defining an infusion portion having at least one opening communicating from the interior of the conduit to the exterior of the conduit through the sidewall, the infusion portion defining a non-linear first shape in its free state such that it tends to return to the first shape after being distorted into a second shape, the first shape defining a generally planar repeating back and forth two-dimensional pattern having a first directional axis corresponding to the pattern length and a second directional axis corresponding to the pattern width.

2. The catheter of claim 1 wherein the first shape comprises:
a generally straight first portion that crosses the length axis generally perpendicular to the length axis;
a generally straight second portion, generally parallel to the first portion, which also crosses the length axis generally perpendicular to the length axis;
a generally straight third portion generally parallel to the second portion which also crosses the length axis generally perpendicular to the length axis;
a first bend connecting the first portion to the second portion; and
a second bend connecting the second portion to the third portion.

3. The catheter of claim 2 wherein the at least one opening is located only in the generally straight portions and not in the bends.

4. The catheter of claim 3 wherein the at least one opening comprises a plurality of openings in each straight portion.

5. The catheter of claim 1 wherein the at least one opening is generally parallel to the generally planar two-dimensional pattern.

6. The catheter of claim 1 wherein the first shape has a generally planar first side and a generally planar second side opposite the first side, the at least one opening communicating through the sidewall between the first and second sides to expel fluid generally parallel to the first and second sides.

7. The catheter of claim 2 wherein the conduit has a transverse conduit dimension transverse to the conduit, the spacing between adjacent portions in the original non-linear shape being between 1 and 100 times the transverse conduit dimension.

8. The catheter of claim 7 wherein the spacing between adjacent portions in the original non-linear shape is between 1 and 20 times the transverse conduit dimension.

9. The catheter of claim 8 wherein the spacing between adjacent portions in the original non-linear shape is between 2 and 5 times the transverse conduit dimension.

10. The catheter of claim 1 wherein the first shape comprises a serpentine shape comprising a regular repeating pattern of two or more curvilinear segments.

11. The catheter of claim 1 wherein the first shape comprises a continuous path of differently shaped curvilinear segments that are bounded by a perimeter defined by the length and width of the pattern.

12. The catheter of claim 11 wherein the segments are generally spaced equal distances from one another.

13. The catheter of claim 11 wherein the perimeter is generally rectangular.

14. The catheter of claim 11 wherein the perimeter is generally elliptical.

15. The catheter of claim 1 further comprising:
a second elongated fluid evacuation conduit having a sidewall and defining an evacuation portion having at least one opening communicating from the exterior of the conduit to the interior of the conduit through the sidewall, the second conduit defining a fluid path from the treatment site to a location outside of the patient.

16. The catheter of claim 15 wherein the second catheter extends beyond a terminal end of the first catheter, the first conduit defining a proximal infusion portion and the second conduit defining a distal evacuation portion.

17. The catheter of claim 16 wherein the first and second conduits are arranged coaxially with the second conduit inside of the first conduit in the proximal infusion portion, the catheter including alternating generally straight portions and bends, the generally straight portions being generally perpendicular to the length axis, the evacuation portion bending back in a direction generally perpendicular to the straight portions to underlie the infusion portion.

18. The catheter of claim 16 wherein the first and second conduits are arranged coaxially with the second conduit inside of the first conduit in the proximal infusion portion, the catheter including alternating generally straight portions and bends, the generally straight portions being generally perpendicular to the length axis, the evacuation portion bending back in a direction generally parallel to the straight portions to underlie the infusion portion.

19. An infusion catheter for delivering treatment fluid to a treatment site of a patient, the catheter comprising:
a plurality of fluid infusion conduits each having a sidewall, each conduit defining a fluid path from a location outside of the patient to the treatment site, each conduit being flexible and defining an infusion portion having at least one opening communicating from the interior of the conduit to the exterior of the conduit through the sidewall, each infusion portion defining a first shape in its free state such that it tends to return to the first shape after being distorted into a second shape, the plurality of conduits being in fluid communication at a first end such that fluid delivered to the first end is distributed to the plurality of infusion portions.

20. The catheter of claim 19 wherein the first shape is selected from the group of shapes consisting of arcs, helixes, serpentine patterns, and polygons.

21. An infusion catheter for delivering treatment fluid to a treatment site of a patient, the catheter comprising:
a fluid infusion conduit having a sidewall and defining a fluid path from a location outside of the patient to the treatment site, the conduit being flexible and defining an infusion portion having at least one opening communicating from the interior of the conduit to the exterior of the conduit through the sidewall, the infusion portion having a non-linear first shape in its free state such that it tends to return to the first shape after being distorted into a second shape, the first shape defining a generally planar spiral coil having a plurality of loops of diminishing size.

22. The catheter of claim 21 wherein the coil has a generally planar first coil side and a generally planar second coil side, the conduit defining a groove formed on a first edge of the conduit and a mating tongue formed on an opposite edge of the conduit such that the when the conduit is in the first, coiled shape the tongue engages the groove to define a barrier between the loops to impede fluid flow from the first coil side to the second coil side.

23. A treatment kit for delivering treatment fluid to a treatment site of a patient, the catheter comprising:
a catheter having a first elongated fluid infusion conduit having a sidewall, the first conduit defining a fluid path from a location outside of the patient to the treatment site, the first conduit being flexible and defining an infusion portion having at least one opening communicating from the interior of the conduit to the exterior of the conduit through the sidewall, the infusion portion defining a non-linear first shape in its free state such that it tends to return to the first shape after being distorted into a second shape, the first shape defining a generally planar repeating back and forth two-dimensional pattern having a first directional axis corresponding to the pattern length and a second directional axis corresponding to the pattern width; and
an introducer defining a lumen through which the conduit can pass when the conduit is distorted into the second shape.

24. A method for delivering treatment fluid to a treatment site of a patient, the method comprising:
providing a catheter having a first elongated fluid infusion conduit having a sidewall, the first conduit defining a fluid path, the first conduit being flexible and defining an infusion portion having at least one opening communicating from the interior of the conduit to the exterior of the conduit through the sidewall, the infusion portion defining a non-linear first shape in its free state such that it tends to return to the first shape after being distorted into a second shape, the first shape defining a generally planar repeating back and forth two-dimensional pattern having a first directional axis corresponding to the pattern length and a second directional axis corresponding to the pattern width;
placing the infusion portion at a treatment site of the patient; and
delivering treatment fluid through the infusion portion to the treatment site.

25. The method of claim 24 wherein placing the infusion portion comprises:
separating tissue layers at the treatment site to create a space between the tissue layers;
placing the non-linear portion of the catheter in the space between the tissue layers; and
transporting fluid to the space between the tissue layers.

26. The method of claim 24 wherein the two-dimensional pattern has a predetermined shape conforming to the margins of the treatment site.

27. The method of claim 24 wherein placing the infusion portion comprises:
distorting the infusion portion into the second shape;
placing the infusion portion at the treatment site; and
allowing the infusion portion to return to the first shape.

28. The method of claim 24 wherein placing the infusion portion comprises:
inserting an introducer from outside the patient into the treatment site, the introducer defining a lumen;
inserting the infusion portion through the lumen and into the treatment site by straightening the infusion portion as it passes through the lumen.

29. The method of claim 24 wherein placing the infusion portion comprises:
inserting an introducer from outside the patient into the treatment site, the introducer defining a lumen;
inserting the infusion portion through the lumen and into the treatment site by folding the infusion portion into a compact configuration as it passes through the lumen.

30. The method of claim 24 wherein placing the infusion portion comprises:
placing the infusion portion at the treatment site to cover a non-linear treatment area;
passing a trocar attached to the first conduit through the skin of the patient; and
passing an end of the first conduit through the skin of the patient.

31. The method of claim 24 wherein placing the infusion portion comprises placing the infusion portion at the treatment site to cover a non-linear treatment area; and
delivering treatment fluid comprises delivering pain relieving medication.

32. The method of claim 24 wherein the catheter further comprises a second elongated fluid evacuation conduit having a sidewall and defining an evacuation portion having at least one opening communicating from the exterior of the conduit to the interior of the conduit through the sidewall, the method further comprising:
placing the evacuation portion at the treatment site.
